(19) United States
(12) Patent Application Publication
(10) Pub. No.: US 2010/0099776 A1
(43) Pub. Date: Apr. 22, 2010

(54) OILY SUSPENSION OF ATOVAQUONE

(75) Inventors: Radhanath MANDAL, East Midnapore (IN); Surajit DAS, Kolkata (IN); Romi Barat SINGH, Varanasi (IN)

Correspondence Address: Ranbaxy Inc., Intellectual Property Department, 600 College Road East, PRINCETON, NJ, 08540, US

(73) Assignee: RANBAXY LABORATORIES LIMITED, Gurgaon (IN)

(21) Appl. No.: 12/582,327

(22) Filed: Oct. 20, 2009

(30) Foreign Application Priority Data
Oct. 20, 2008 (IN) 2394/DEL/2008

(51) Int. Cl. A61K 31/122; A61P 31/02

(52) U.S. Cl. 514/682

(57) ABSTRACT

The present invention relates to an oily suspension of atovaquone comprising atovaquone particles and a combination of surfactants having HLB more than 10.

### FIELD OF THE INVENTION

The present invention relates to an oily suspension of atovaquone comprising atovaquone particles and a combination of surfactants having HLB more than 10.

### BACKGROUND OF THE INVENTION

Many orally-administered drugs display poor bioavailability when administered in conventional dosage forms. With several drugs, absorption may be as little as 30 percent or less of the orally administered dose. To compensate for this effect, a very large dose is often administered so that absorption of the therapeutically required quantity of the drug can occur.

This technique may prove costly with expensive drugs, and the non-absorbed drug may also have undesirable side effects within the gastrointestinal tract. In addition, poorly absorbed drugs often display a great deal of inter-patient variability in bioavailability, and this can create dosing problems. Poor bioavailability is often associated with poor solubility of drugs. There are various techniques available to overcome solubility and bioavailability problem, and one such viable technique is particle size reduction. Lipid based formulations such as micelles, emulsions, or SEDDS etc. can also enhance the bioavailability of poorly water-insoluble drugs. Self-emulsifying drug delivery systems (SEDDS) form fine oil-in-water (O/W) emulsions, with a particle size significantly smaller than in conventional emulsions, when introduced into aqueous media under gentle agitation thus preventing drug precipitation and hence improving drug absorption.

Atovaquone, 244-(4-chlorophenyl)cyclohexyl′-3-hydroxy-1,4-naphthoquinone is a widely used antiprotozoal and is potently active (in animals and in vitro) against Pneumocystis carinii, Plasmodia, and tachyzoite and cyst forms of Toxoplasma gondii. Atovaquone, a highly lipophilic compound resembling ubiquinone, has a low aqueous solubility, and that is the reason for the poor bioavailability of atovaquone after oral administration. It is reported that after a single oral dose, absorption of the drug is slow and erratic, and that it increases about three-fold by the presence of fatty food and is dose-limited above 750 mg.

U.S. Pat. No. 4,981,874 discloses the use of atovaquone against Pneumocystis carinii infection in a mammal. European Patent No. EP 0 123 238 and U.S. Pat. No. 5,053,432 discloses the use of atovaquone against Plasmodium falciparum and also against Eimeria species such as E. tenella and E. acervulina which are causative organisms of coccidiosis. Further, use of atovaquone against Toxoplasmosis and Cryptosporidiosis is disclosed in European Patent Nos. EP 0 445 141 and EP 0 496 729, respectively.

Currently, atovaquone suspension marketed under trade name MEPRON® is an aqueous suspension comprising micro-fine particles of atovaquone. Atovaquone particles are reduced in size to facilitate absorption. These particles are significantly smaller than those in the previously marketed tablet formulation. Further, U.S. Pat. Nos. 6,018,080 and 6,649,659 discloses microfluidized particles of atovaquone having improved bioavailability, wherein at least 90% of atovaquone particles have a volume diameter in the range of 0.1-3 micron.

### SUMMARY OF THE INVENTION

The present invention relates to an oily suspension of atovaquone comprising atovaquone particles and a combination of surfactants having hydrophilic-lipophilic balance (HLB) of more than 10.

Hence, according to one of the aspects, there is provided an oily suspension of atovaquone comprising
a) atovaquone particles; and
b) a combination of surfactants having HLB more than 10.

In another aspect, there is provided an oily suspension of atovaquone comprising
a) atovaquone particles; and
b) a combination of surfactants comprising PEG8 caprylic/capric glycerides and polyoxyl 35 castor oil.

In another aspect, there is provided an oily suspension of atovaquone comprising
a) atovaquone particles; and
b) a combination of surfactants having HLB more than 10
   wherein the particles have d₉₀ value of about 4-15 μm.

In another aspect, there is provided a process of preparation of an oily suspension of atovaquone comprising the steps of:
a) heating a portion of oil and dissolving surfactant mixture in it,
b) dispersing atovaquone in solution of step a),
c) heating another portion of oil and dissolving other pharmaceutically acceptable excipients,
d) adding slowly atovaquone dispersion of step b) to step c) under continuous stirring; and
e) stirring/homogenizing the dispersion of step d) till a uniform suspension is obtained and filling into suitable size bottles.

In another aspect, there is provided a method for the treatment of protozoal infection, the method comprising: orally administering to a subject an oily suspension of atovaquone comprising atovaquone particles and a combination of surfactants having HLB more than 10.

### DETAILED DESCRIPTION OF THE INVENTION

“Atovaquone” as employed herein is intended to include isomers, cis and trans forms of atovaquone or mixture thereof or any pharmaceutically acceptable salts thereof. Atovaquone may be used in any of the Polymorphic forms such as Form I or III. It may be used alone or in combination with proguanil.

As used herein, the term “pharmaceutically acceptable salts” refers to inorganic base salts such as alkali metal (e.g., sodium and potassium) salts and alkaline earth metal (e.g., calcium) salts; organic base salts e.g. phenylethylbenzylamine, dibenzylethylenediamine, ethanolamine and diethanolamine salts; and amino acid salts, e.g., lysine and arginine.

The term “oily suspension” refers to an oil-based liquid suspension for delivering a medicament. The oily suspension may comprise oil or oily substance or mixture as a carrier medium. The oils are selected from the group consisting of animal oils such as mink oil, squalene; vegetable oils; hydrogenated vegetable oils and mineral oils such as liquid paraffin. Vegetable oils include oils such as coconut oil, canola oil, cottonseed oil, olive oil, palm oil, corn oil, sesame oil, safflower oil, avocado oil and soybean oil. The oily substance may be selected from medium chain triglycerides, long chain triglycerides, and mixtures of mono-, di- and tri-glycerides of fatty acids. Examples of oily substance include MIGLYOL® 812 which is a 56% caprylic (C8) and 36% capric (C10) triglyceride, MIGLYOL 810 (68% C8 and 28% C10), NEOBEE® M5, CAPTEX® 300, CAPTEX 355, LABRAFAC CRODAMOL GTCC™, SOFTISANS™ 100, SOFTISANS 142, SOFTISANS 378, and SOFTISANS 649.

The term “HLB” refers to hydrophilic-lipophilic balance of a molecule. The HLB number increases with increasing hydrophilicity. That is, the higher the HLB number, the more hydrophilic the surfactant or emulsifying agent. In the present invention, the preferred HLB value for the surfactants is more than 10.

The surfactants with HLB more than 10 may be selected from the group consisting of sodium lauryl sulfate (SLS), polyoxyethylene sorbitan fatty acid esters such as polyoxyethylene sorbitan monolaurate, polyoxyethylene sorbitan monopalmitate, polyoxyethylene sorbitan monostearate, polyoxyethylene sorbitan tristearate; dialkyl sodium sulfosuccinates such as bis-(2-ethylhexyl) sodium sulfosucinate, polyglycolized glycerides such as GELUCIRE® and LABRASOL®; polyoxyethylene-polyoxypropylene block co-polymer such as poloxamers; fatty acid esters of polyethylene glycol such as PEG 600 monooleate, PEG 400 dioleate, PEG 600 dioleate, PEG 400 monostearate, PEG 600 monostearate, PEG 8000 distearate, PEG 10000 distearate, PEG 200 monolaurate, PEG 400 monolaurate and PEG 600 monolaurate; polyoxyethylene alkyl ethers such as BRIJ®, particularly BRIJ-35 and BRIJ-700, and Cremophors; polyoxyethylene fatty acid esters include polyoxyethylene stearates such as MYRJ®.

Cremophors (Polyoxyl 35 castor oil) are non-ionic emulsifiers obtained by causing ethylene oxide to react with hydrogenated castor oil particularly in a molar ratio of about 35 moles to 1 mole. Polyethoxylated castor oil known as CREMOPHOR® is available in different grades such as CREMOPHOR A6, CREMOPHOR A25, CREMOPHOR RH 410, CREMOPHOR RH 40, CREMOPHOR RH 60, CREMOPHOR CO 40, CREMOPHOR CO 410, CREMOPHOR CO 455, CREMOPHOR CO 60, CREMOPHOR EL. The amount of CREMOPHOR may vary from 1-70 mg/ml.

The polyglycolyzed glyceride may be saturated or unsaturated and include ethoxylated glycerides and polyethylene glycol esters. The HLB value of the polyglycolized glycerides is adjusted by the length of the PEG chain and the melting point is adjusted by the length of the chains of the fatty acids, of the PEG and by the degree of saturation of the fatty chains. A particularly preferred polyglycolyzed glyceride is a glyceryl caprylate/caprate and PEG-8 (polyethylene glycol) caprylate/caprate complex known as LABRASOL®. The amount of LABRASOL may vary from 1-30 mg/ml.

The term “d₉₀ value” means at least 90% of atovaquone particles have volume diameter less than specified value when measured by a light scattering method, for example, Malvern Mastersizer. Atovaquone particles have d₉₀ value of about 4-15 μm. In particular, the particle size of atovaquone is d₉₀ value 4-9 μm and corresponding d₅₀ value of 1-5 μm.

Particle size reduction may be carried out using various conventionally available mills such as ball mill, an attritor mill, a vibratory mill, air jet mill and media mills such as a sand mill and a bead mill. Air jet mill can be used only for dry milling process whereas all the other mills may be used for both dry as well as wet milling. The milling may be carried out using the atovaquone alone or with other pharmaceutically acceptable excipients such as surfactants or binding agents or a diluent.

The suspension dosage form should have suitable properties such as viscosity, taste and flavor. The pharmaceutically acceptable excipients may be selected from suspending agent, solvents, preservatives, oils, coloring agents, antioxidants, flavoring agents and sweeteners.

Suspending agent is selected from the group consisting of polysaccharide, (tragacanth; xanthan gum; bentonite; acacia and lower alkyl ethers of cellulose (including the hydroxy and carboxy derivatives of the cellulose ethers)), vinyl polymers such as povidone, a mixture of cellulose and of xanthan gum, a mixture of polyethylene glycol and of sodium carboxymethyl cellulose, a mixture of xanthan gum and of pregelatinized starch, a mixture of microcrystalline cellulose and of sodium carboxymethyl cellulose (AVICEL® RC 591), dispersed silicon dioxide (AEROSIL® 200). The amount of suspending agent may range from 0.01-5% w/v.

The preservatives may be selected from benzyl alcohol, propylparaben, methylparaben, sorbic acid, sodium benzoate and sodium bisulphate.

The antioxidants may be selected from butylated hydroxy anisole (BHA), butylated hydroxy toluene (BHT), lipoic acid, lutein, lycophyll, xanthophyll, carotene, vitamin E and esters thereof, sulfurous acid salts such as sodium sulfate, sodium bisulfite, sodium metabisulfite, sodium sulfite, sodium formaldehyde sulfoxylate, and sodium thiosulfate, Nordihydroguaiaretic acid.

Sweeteners may be selected from sucrose, lactose, glucose, sucralose, acesulfame potassium, aspartame, saccharine, and sorbitol solution.

The suitable flavoring agents may be selected from yellow plum lemon, tutti frutti, aroma, peppermint oil, oil of wintergreen, cherry, orange or raspberry flavor.

According to one of the embodiment, process for the preparation of oily suspension of atovaquone comprises the steps of
a) heating a portion of oil under stirring till a clear solution is obtained and adding preservative and antioxidant to the clear solution;
b) heating second portion of oil and dissolving a surfactant mixture of PEG-8 caprylic/capric glycerides and Polyoxyl 35 castor oil in it;
c) dispersing atovaquone in solution of step b);
d) heating third portion of oil and dissolving/dispersing other pharmaceutically acceptable excipients in it;
e) adding the dispersion of step d) to the solution of step a);
f) adding slowly atovaquone dispersion of step c) to step e) under continuous stirring; and
g) homogenizing the dispersion of step f) till a uniform suspension is obtained and filling into suitable size bottles.

The following example is provided to enable one of ordinary skill in the art to prepare dosage forms of the invention and should not be construed as limiting the scope of invention.

### EXAMPLES

### Example 1

| | | Qty |
|---|---|---|
| S No. | Ingredients | (mg/5 mL) |
| 1 | Atovaquone | 750.00 |
| 2 | CREMOPHOR EL | 299.50 |
| 3 | LABRASOL | 35.00 |
| 4 | Hydrogenated vegetable oil | 125.00 |
| 5 | CAPTEX 300 | 120.00 |
| 6 | Saccharin Sodium | 35.00 |
| 7 | Colloidal silicon dioxide | 40.00 |
| 8 | Butylated hydroxyl anisole | 0.50 |
| 9 | Methyl paraben | 1.00 |
| 10 | Propyl paraben | 0.10 |
| 11 | Tutti frutti flavor | 25.00 |
| 12 | Liquid paraffin | Q.S to 5 mL |

### Procedure:

1. A part of liquid paraffin was heated and hydrogenated vegetable oil was added under stiffing till a clear solution was obtained.
2. Methyl paraben and propyl paraben were dissolved in solution of step 1.
3. Butyl hydroxyanisole was added to the solution of step 2
4. Another portion of liquid paraffin was heated and CREMOPHOR® EL, LABRASOL® and CAPTEX® were dissolved in it.
5. Atovaquone was dispersed in a solution of step 4.
6. Remaining liquid paraffin was heated and colloidal silicon dioxide was dissolved in it.
7. Saccharin sodium was dispersed in solution of step 6.
8. The dispersion of step 7 was added into solution of step 3.
9. Atovaquone dispersion of step 5 was slowly added to step 8 under continuous stiffing.
10. Flavors were added to dispersion of step 8 and final volume was made up with remaining amount of liquid paraffin.
11. The dispersion was homogenized for a suitable period of time till a uniform suspension was obtained.

We claim:

1. An oily suspension of atovaquone comprising:
a) atovaquone particles; and
b) a combination of surfactants having HLB more than 10.

2. The oily suspension of atovaquone according to claim 1 wherein the atovaquone particles have d₉₀ value of about 4-15 μm.

3. The oily suspension of atovaquone according to claim 1 wherein the surfactants are selected from the group consisting of sodium lauryl sulfate (SLS), polyoxyethylene sorbitan fatty acid esters, dialkyl sodium sulfosuccinates, polyglycolized glycerides; polyoxyethylene-polyoxypropylene block co-polymer; polyoxyethylene alkyl ethers, fatty acid esters of polyethylene glycol, and polyoxyethylene fatty acid esters or mixtures thereof.

4. The oily suspension of atovaquone according to claim 3 wherein the surfactant is a combination of polyethoxylated castor oil and polyglycolized glycerides.

5. The oily suspension of atovaquone according to claim 4 wherein polyethoxylated castor oil is Polyoxyl 35 castor oil.

6. The oily suspension of atovaquone according to claim 4 wherein polyglycolized glycerides is PEG 8 caprylic/capric glyceride.

7. The oily suspension of atovaquone according to claim 1 wherein the suspension further comprises one or more pharmaceutically acceptable excipients selected from the group consisting of oil, oily substance, preservatives, antioxidants, suspending agents, coloring agents, flavoring agents and sweeteners.

8. The oily suspension of atovaquone according to claim 7 wherein the oils are selected from the group consisting of animal oils, vegetable oils, hydrogenated vegetable oils and mineral oils or mixtures thereof.

9. The oily suspension of atovaquone according to claim 7 wherein the oily substance is selected from medium chain triglycerides, mono-, di- and tri-glycerides of fatty acids, long chain triglycerides or mixtures thereof.

10. The oily suspension of atovaquone according to claim 1 wherein the suspension is prepared by a process comprising the steps of:
a) heating a portion of oil and dissolving surfactant mixture in it,
b) dispersing atovaquone in solution of step a),
c) heating another portion of oil and dissolving other pharmaceutically acceptable excipients,
d) adding slowly atovaquone dispersion of step b) to step c) under continuous stirring; and
e) stirring/homogenizing the dispersion of step d) till a uniform suspension is obtained and filling into suitable size bottles.
